(19) United States
(12) Patent Application Publication
(10) Pub. No.: US 2026/0166215 A1
(43) Pub. Date: Jun. 18, 2026

(54) NEGATIVE PRESSURE WOUND THERAPY SYSTEM FOR ASSISTING IN MANAGING THE USE OF WOUND DRESSINGS

(71) Applicant: Fortune Medical Instrument Corp., New Taipei City (TW)

(72) Inventors: TAI-LI WANG, New Taipei City (TW); Wei-Chiang Hsu, New Taipei City (TW); Chia-Wen Ko, New Taipei City (TW)

(21) Appl. No.: 19/405,481

(22) Filed: Dec. 2, 2025

Related U.S. Application Data
(60) Provisional application No. 63/733,455, filed on Dec. 13, 2024.

(30) Foreign Application Priority Data
Nov. 4, 2025 (TW) 114142955

(51) Int. Cl. A61M 1/00

(52) U.S. Cl. CPC A61M 1/98; A61M 2205/3324; A61M 2205/3327; A61M 2205/3331; A61M 2205/3368; A61M 2205/502; A61M 2205/52; A61M 2205/60

(57) ABSTRACT

A negative pressure wound therapy system for assisting in managing the use of wound dressing includes a wound dressing applied to a wound; a storage medium disposed on the wound dressing and storing wound dressing information; a catheter connected to the wound dressing; a collection device connected to the catheter; a vacuum pump; a monitoring device communicatively connected to the storage medium; and a tubing extending through the monitoring device and connected to the collection device and the vacuum pump. The monitoring device receives the wound dressing information and based on the wound dressing information, determines whether the wound dressing has been used for treatment. If it is determined as yes, the monitoring device displays that the wound dressing has been repeatedly used; otherwise, the monitoring device displays that the wound dressing is used for the first time.

### CROSS-REFERENCE TO RELATED APPLICATION

This application claims the benefit of the U.S. Provisional Application No. 63/733,455, filed on Dec. 13, 2024, and Taiwan Patent Application No. 114142955, filed on Nov. 4, 2025, which are hereby incorporated by reference as if fully set forth herein.

### BACKGROUND OF THE INVENTION

### 1. Field of the Invention

The present invention relates to a negative pressure wound therapy system, in particular a negative pressure wound therapy system for assisting in managing the use of wound dressings.

### 2. The Prior Art

Negative pressure wound therapy is an assisting therapy method that creates a negative pressure environment to help wound healing. Its principle is to maintain a certain vacuum state between the wound and the dressing through negative pressure to achieve the following effects: first, removing wound exudate and infected tissue, reducing bacterial reproduction; secondly, promoting blood circulation and reducing edema; thirdly, stimulating the growth of granulation tissue; and fourth, keeping the wound moist.

Conventional negative pressure wound therapy systems include a wound dressing, a catheter, a collection device, a vacuum pump, and a tubing. The wound dressing is placed on a wound, the catheter is connected to the wound dressing, the collection device is connected to the catheter, and the tubing is connected to the collection device and the vacuum pump. When the vacuum pump is turned on, it draws air from the collection device through the tubing, creating a vacuum and providing negative pressure. The wound dressing uses the negative pressure to draw fluid drainage from the wound through the catheter into the collection device, achieving a drainage effect so as to heal the wound.

However, the conventional negative pressure wound therapy systems do not manage the use of wound dressings. Therefore, health care workers need to confirm the wound condition and record the use of wound dressings. However, it is prone to human error, such as repeated use of wound dressings or failure to replace wound dressings, which can lead to wound deterioration.

Furthermore, the wound dressing includes a foam pad and an adhesive film. The foam pad is placed in the wound, and the adhesive film covers the foam pad and the wound and adheres closely to the normal skin to form a closed wound environment. Under the normal use of the wound dressing, the foam pad easily covers the wound. It's difficult for the health care workers to directly observe the wound during the negative pressure wound therapy and therefore unable to monitor the real-time status of the wound environment at any time. Therefore, the health care workers can observe the wound's condition only when the wound dressing is removed during dressing replacements.

### SUMMARY OF THE INVENTION

The primary objective of the present invention is to provide a negative pressure wound therapy system for assisting in managing the use of wound dressings, which can determine whether the wound dressing has been used for treatment.

Another objective of the present invention is to provide a negative pressure wound therapy system for assisting in managing the use of wound dressings, which can calculate the usage time of the wound dressings.

Another objective of the present invention is to provide a negative pressure wound therapy system for assisting in managing the use of wound dressings, which can monitor the real-time status of the wound environment at any time.

In order to achieve the aforementioned objectives, the present invention provides a negative pressure wound therapy system for assisting in managing the use of wound dressings, comprising a wound dressing, a storage medium, a catheter, a collection device, a vacuum pump, a monitoring device, and a tubing. The wound dressing is used to be disposed on a wound. The storage medium is disposed on the wound dressing and is used to store wound dressing information. The catheter is connected to the wound dressing. The collection device is connected to the catheter. The monitoring device is communicatively connected to the storage medium. The tubing extends through the monitoring device, one end of the tubing is connected to the collection device, and another end of the tubing is connected to the vacuum pump. The monitoring device receives the wound dressing information, and the monitoring device determines whether the wound dressing has been used for treatment based on the wound dressing information. If it is determined as yes, the monitoring device displays that the wound dressing has been repeatedly used. If it is determined as no, the monitoring device displays that the wound dressing is used for the first time.

According to an embodiment, if it is determined as no, the monitoring device starts to calculate the usage time of the wound dressing and displays the usage time.

According to an embodiment, the storage medium receives the usage time and integrates the usage time into the wound dressing information.

According to an embodiment, the wound dressing information includes an identification code, which includes at least a serial number, a batch number, a model number, or a type of the wound dressing. The monitoring device determines whether the wound dressing has been used for treatment based on the identification code and displays the identification code.

According to an embodiment, the negative pressure wound therapy system further comprises a position sensor. The position sensor is disposed in the wound dressing, communicatively connected to the monitoring device, and used to detect a relative position of the wound dressing and the monitoring device to generate position information, wherein the monitoring device receives the position information and displays adjusting position information of the monitoring device according to the position information.

According to an embodiment, the negative pressure wound therapy system further comprises a wound environment sensor. The wound environment sensor is disposed in the wound dressing, communicatively connected to the monitoring device, and used to detect a wound environment parameter, wherein the monitoring device receives the wound environment parameter and displays the wound environment parameter.

According to an embodiment, the collection device, the vacuum pump, and the monitoring device are separately provided.

According to an embodiment, the collection device is integrally formed on a side of the monitoring device, and the vacuum pump and the monitoring device are separately provided.

According to an embodiment, the collection device is integrally formed on a side of the monitoring device, and the vacuum pump is disposed in the monitoring device.

According to an embodiment, the collection device and the monitoring device are separately provided, and the vacuum pump is disposed in the monitoring device.

The effect of the present invention is that when the monitoring device indicates that the wound dressing has been repeatedly used, the health care workers can observe and know from the monitoring device that the wound dressing has not been replaced, and can quickly remove the used wound dressing and place a new wound dressing on the wound to prevent the wound from deterioration. When the monitoring device indicates that the wound dressing is used for the first time, the health care workers can observe and know from the monitoring device that the wound dressing has been replaced, avoiding premature replacement of the wound dressing, maximizing the efficacy of the wound dressing, and reducing the waste of wound dressing.

Furthermore, the health care workers can observe the usage time of the wound dressing from the monitoring device, which helps determine whether the wound dressing needs to be replaced. If the usage time of the wound dressing is greater than a predetermined time, the health care workers will immediately replace the wound dressing to prevent the wound from deterioration. If the usage time of the wound dressing is less than the predetermined time, the health care workers will not replace the wound dressing, avoiding premature replacement of the wound dressing to maximize the efficacy of the wound dressing, and reduce waste of wound dressing.

In addition, the health care workers can observe wound environment parameters from the monitoring device and determine the wound condition based on the wound environment parameters, achieving the effect of monitoring the real-time status of the wound environment at any time, without having to remove the wound dressing to observe the wound condition during dressing replacements.

### BRIEF DESCRIPTION OF THE DRAWINGS

FIG. 1 is a schematic view of a first embodiment of the present invention;

FIG. 2 is a schematic view showing the connection relationship between a storage medium, a monitoring device, a position sensor, and a wound environment sensor according to the first embodiment of the present invention;

FIG. 3 is a schematic view of the monitoring device of the first embodiment of the present invention displaying the wound environment parameters;

FIG. 4 is a schematic view of a second embodiment of the present invention;

FIG. 5 is a schematic view of a third embodiment of the present invention; and

FIG. 6 is a schematic view of a fourth embodiment of the present invention.

### DETAILED DESCRIPTION OF THE PREFERRED EMBODIMENT

The detailed description of the embodiments hereinafter is for describing the principle of the present invention in an exemplary manner, but not for limiting the scope of the present invention. The present invention may be embodied in various and different forms, and is not limited to certain embodiments disclosed herein, but comprises all technical solutions falling within the scope of claims.

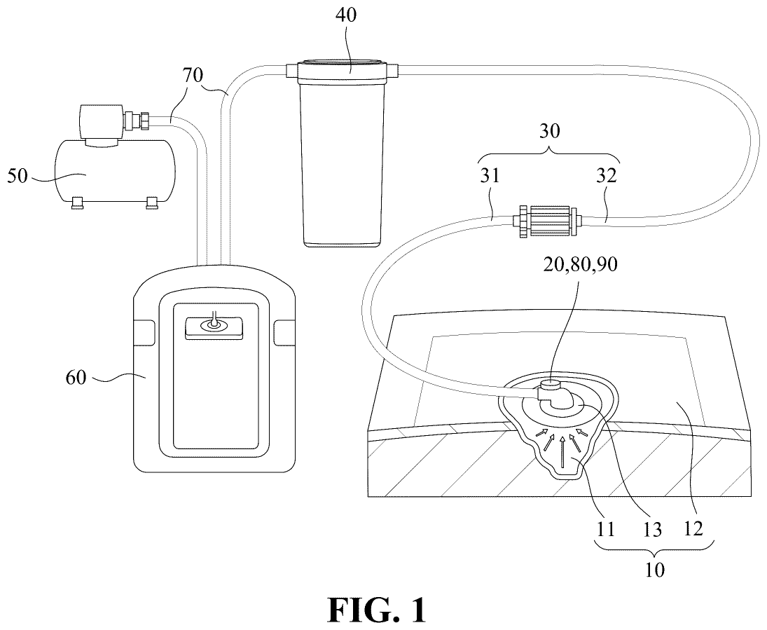

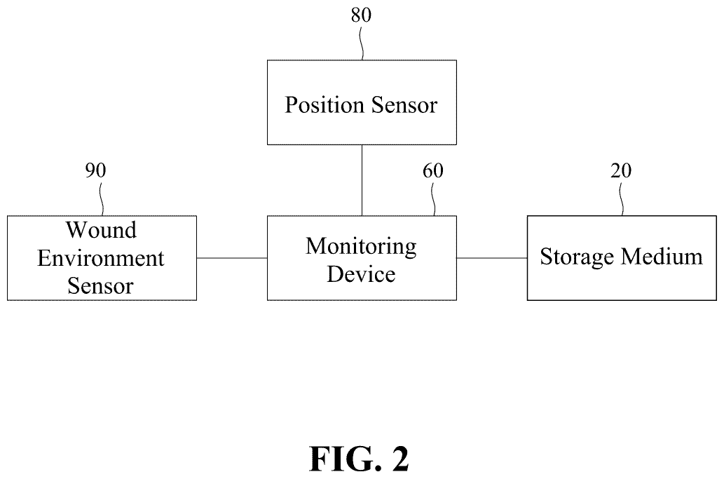

FIG. 1 is a schematic view of the first embodiment of the present invention. FIG. 2 is a schematic view of the connection relationship between a storage medium 20, a monitoring device 60, a position sensor 80, and a wound environment sensor 90 of the first embodiment of the present invention. As shown in FIGS. 1 and 2, the present invention provides a negative pressure wound therapy system for assisting in managing the use of a wound dressing 10, comprising a wound dressing 10, a storage medium 20, a catheter 30, a collection device 40, a vacuum pump 50, a monitoring device 60, and a tubing 70. The wound dressing 10 is configured to be applied to a wound. The storage medium 20 is disposed on the wound dressing 10 and is used to store wound dressing information. The catheter 30 is connected to the wound dressing 10, the collection device 40 is connected to the catheter 30, the monitoring device 60 is communicatively connected to the storage medium 20, and the tubing 70 extends through the monitoring device 60. One end of the tubing 70 is connected to the collection device 40, and another end of the tubing 70 is connected to the vacuum pump 50.

As shown in FIG. 1, the vacuum pump 50 is turned on. The vacuum pump 50 draws air from the collection device 40 through the tubing 70 to create a vacuum and provide negative pressure. The wound dressing 10 uses the negative pressure to draw fluid drainage from the wound through the catheter 30 into the collection device 40, achieving a drainage effect so as to heal the wound.

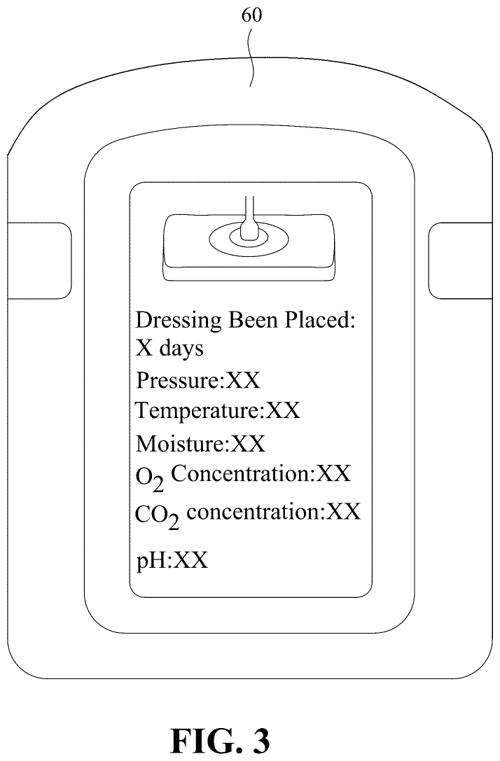

FIG. 3 is a schematic view of the monitoring device 60 displaying wound environment parameters according to a first embodiment of the present invention. As shown in FIG. 3, the monitoring device 60 receives the wound dressing information and determines whether the wound dressing 10 has been used for treatment based on the wound dressing information. If it is determined as yes, the monitoring device 60 displays that the wound dressing 10 has been repeatedly used. If it is determined as no, the monitoring device 60 displays that the wound dressing 10 is used for the first time. When the monitoring device 60 indicates that the wound dressing 10 has been repeatedly used, the health care workers can observe and know from the monitoring device 60 that the wound dressing 10 has not been replaced, and can quickly remove the used wound dressing 10 and place a new wound dressing 10 on the wound to prevent the wound from deterioration. When the monitoring device 60 indicates that the wound dressing 10 is used for the first time, the health care workers can observe and know from the monitoring device 60 that the wound dressing 10 has been replaced, avoiding premature replacement of the wound dressing 10, maximizing the efficacy of the wound dressing 10, and reducing waste of the wound dressing 10.

As shown in FIG. 1, according to the first embodiment, the wound dressing 10 includes a foam pad 11, an adhesive film 12 and a suction disk 13. The foam pad 11 is placed in the wound, and the adhesive film 12 covers the foam pad 11 and the wound and adheres closely to the normal skin to form a closed wound environment. Preferably, the adhesive film 12 is made of polyurethane, but the present invention is not limited to this. An opening is cut on the adhesive film 12, and the opening is located above the closed wound. The suction disk 13 is disposed above the opening to achieve an airtight effect. The catheter 30 is connected to the suction disk 13, and the suction disk 13 draws the fluid drainage from the wound into the collection device 40 through the catheter 30 by negative pressure.

The storage medium 20 may be, for example, any type of static or removable random access memory (RAM), read-only memory (ROM), flash memory, a hard disk drive (HDD), a solid state drive (SSD), or similar devices or a combination thereof. The communication connection may be a wired or wireless transmission method. The wired transmission method includes a transmission line (not shown) extending within the catheter 30. The wireless transmission method includes Bluetooth, near field communication, radio frequency communication, Wi-Fi, infrared, wireless area network, 5G mobile network, 5G new radio, long term evolution network, 3G mobile network, 2G mobile network, or wireless transmission technology specified by the Global System for Mobile Communications. However, the present invention is not limited thereto.

As shown in FIG. 1, according to the first embodiment, the catheter 30 includes two pipes 31 and 32. One end of the pipe 31 is connected to the suction disk 13, and another end of the pipe 31 is connected to one end of the pipe 32 by means of a snap or threads. Another end of the pipe 32 is connected to the collection device 40. The collection device 40, the vacuum pump 50, and the monitoring device 60 are separately provided.

As shown in FIG. 3, according to the first embodiment, the monitoring device 60 receives the wound dressing information. The monitoring device 60 determines whether the wound dressing 10 has been used for treatment based on the wound dressing information. If it is determined as no, the monitoring device 60 starts to calculate the usage time of the wound dressing 10 and displays the usage time. In this way, the health care workers can observe and know the usage time of the wound dressing 10 from the monitoring device 60 to help determine whether to replace the wound dressing 10. If the usage time of the wound dressing 10 is more than a predetermined time, the health care workers will immediately replace the wound dressing 10 to prevent the wound from deterioration. If the usage time of the wound dressing 10 is less than the predetermined time, the health care workers will not replace the wound dressing 10, avoiding premature replacement of the wound dressing 10, maximizing the efficacy of the wound dressing 10, and reducing waste of the wound dressing 10.

Preferably, the usage time can be minutes, hours, days or a combination thereof.

As shown in FIG. 2, according to the first embodiment, the storage medium 20 receives the usage time and integrates it into the wound dressing information. Thus, the storage medium 20 can record the usage condition of the wound dressing 10. When the health care workers want to know the usage time of the wound dressing 10 for the previous applications, they can use the monitoring device 60 to retrieve the historical records of the wound dressing information from the storage medium 20, which helps the health care workers understand whether the replacement frequency of the wound dressing 10 is stable.

According to the first embodiment, the wound dressing information includes an identification code, which includes at least a serial number, a batch number, a model number or a type of the wound dressing 10. The monitoring device 60 determines whether the wound dressing 10 has been used for treatment based on the identification code and displays the identification code at the same time. The health care workers can observe and know the serial number, the batch number, the model number or the type of the wound dressing 10 from the monitoring device 60.

As shown in FIGS. 1 and 2, according to the first embodiment, the negative pressure wound therapy system of the present invention further includes a position sensor 80. The position sensor 80 is disposed in the wound dressing 10, is communicatively connected to the monitoring device 60, and is used to detect the relative position of the wound dressing 10 and the monitoring device 60 to generate position information. The monitoring device 60 receives the position information and displays adjusting position information of the monitoring device 60 based on the position information. For example, when the position of the monitoring device 60 is higher than the position of the wound dressing 10, the adjusting position information is “It is recommended to position the monitoring device 60 lower than the wound dressing 10”, so as to establish a good wound environment.

As shown in FIGS. 1 and 2, according to the first embodiment, the negative pressure wound therapy system of the present invention further includes a wound environment sensor 90. The wound environment sensor 90 is disposed in the wound dressing 10, is communicatively connected to the monitoring device 60, and is used to detect a wound environment parameter. As shown in FIG. 3, the monitoring device 60 receives the wound environment parameters and displays the wound environment parameters. Accordingly, the health care workers can observe and know the wound environment parameter from the monitoring device 60 and determine the wound condition based on the wound environment parameter, achieving the effect of monitoring the real-time status of the wound environment at any time, without having to remove the wound dressing 10 to observe the wound condition during dressing replacements.

Preferably, the wound environment sensor 90 includes a pressure sensor, a temperature sensor, a moisture sensor, an oxygen concentration sensor, a carbon dioxide concentration sensor or a pH sensor; and the wound environment parameters include a pressure, a temperature, a moisture, an oxygen concentration, a carbon dioxide concentration or a pH value. The following will describe more detailed how the various wound environment sensors 90 operate.

When the vacuum pump 50 is turned on, the vacuum pump 50 provides a negative pressure to the collection device 40, the pressure sensor detects the wound environment pressure, the monitoring device 60 receives the wound environment pressure and displays the wound environment pressure, and the health care workers can observe and know the wound environment pressure from the monitoring device 60 to determine whether the wound environment pressure is leaking and whether the pressure is stable. When the wound environment pressure is lower than a certain range of the preset pressure, for example, the wound environment pressure is 115 mmHg, the preset pressure is 125 mmHg, and the wound environment pressure is significantly lower than the preset pressure by about 10 mmHg, the vacuum pump 50 will adjust the pressure of the negative pressure provided to the collection device 40. When the wound environment pressure is greater than or equal to the preset pressure, the vacuum pump 50 is turned off, and the vacuum pump 50 stops providing negative pressure to the collection device 40. When the wound environment pressure is lower than the preset pressure again, the vacuum pump 50 is turned on again.

The temperature sensor detects the wound environment temperature. The monitoring device 60 receives the wound environment temperature and displays the wound environment temperature. When the wound environment temperature is greater than the preset high temperature or is lower than the preset low temperature, for example, the preset high temperature is 37.5° C. and the preset low temperature is 30.2° C., the health care workers can observe and know from the monitoring device 60 that the wound environment temperature is greater than the preset high temperature or is lower than the preset low temperature, and determine that the wound temperature of a patient is abnormal, and recommend checking the patient's wound condition or the patient's overall condition. The wound environment temperature can be used in the monitoring device 60 for short-term monitoring, for example, by observing the changes in the wound environment temperature, reminding the health care workers to check the patient's wound condition.

The moisture sensor detects the wound environment moisture. The monitoring device 60 receives the wound environment moisture and displays it. When the wound environment moisture is lower than the preset moisture, for example, the preset moisture is 70% RH, the health care workers can observe and know from the monitoring device 60 that the wound environment moisture is lower than the preset moisture, and determine that the wound is relatively dry, and can recommend an assisting therapy, such as using perfusion therapy, to establish a good wound environment. Perfusion therapy refers to further adding a perfusion process during the negative pressure therapy, applying the perfusion fluid to the wound, which may include soaking the wound in liquid for a short time, and then continuing the negative pressure therapy process.

The oxygen concentration sensor detects the oxygen concentration in the wound environment. The monitoring device 60 receives the oxygen concentration in the wound environment and displays it. When the oxygen concentration in the wound environment is lower than the oxygen concentration in the surrounding environment, the health care workers can observe and know from the monitoring device 60 that the oxygen concentration in the wound environment is lower than the oxygen concentration in the surrounding environment, and determine that the oxygen concentration in the patient's wound environment is too low. Assisting therapy, such as local hyperoxia therapy, is recommended to establish a good wound environment. Local hyperoxia therapy refers to the assisting therapy of the wound with high-concentration oxygen (>21% oxygen).

The carbon dioxide concentration sensor detects the carbon dioxide concentration in the wound environment. The monitoring device 60 receives the carbon dioxide concentration in the wound environment and displays it. When the carbon dioxide concentration in the wound environment is lower than the carbon dioxide concentration in the surrounding environment, the health care workers can observe and know from the monitoring device 60 that the carbon dioxide concentration in the wound environment is lower than the carbon dioxide concentration in the surrounding environment, and determine that the carbon dioxide concentration in the patient's wound environment is too low, and can recommend assisting therapy, such as local carbon dioxide therapy, to establish a good wound environment. Local carbon dioxide therapy refers to the application of high-concentration carbon dioxide to the wound for assisting therapy.

The pH sensor detects the pH value of the wound environment. The monitoring device 60 receives the pH value of the wound environment and displays it. When the pH value of the wound environment is greater than the preset pH value, for example, the preset pH value is 8.9, the health care workers can observe and know from the monitoring device 60 that the pH value of the wound environment is greater than the preset pH value, and determine that the pH value of the patient's wound environment is too high, and can recommend an assisting therapy, such as adjusting the pH value of the wound to establish a good wound environment. The pH value of the wound environment can be used in the monitoring device 60 for short-term monitoring, for example, by observing the changes in the pH value of the wound environment, reminding the health care workers to observe the patient's wound healing status.

As shown in FIGS. 1 and 2, a plurality of wound environment sensors 90 of different types, such as a pressure sensor, a temperature sensor, a moisture sensor, an oxygen concentration sensor, a carbon dioxide concentration sensor, and a pH sensor, can be simultaneously provided in the wound dressing 10. These wound environment sensors 90 are responsible for detecting different wound environment parameters such as pressure, temperature, moisture, oxygen concentration, carbon dioxide concentration, and pH value. The monitoring device 60 receives the plurality of wound environment parameters and displays the wound environment parameters, providing the health care workers with a comprehensive assessment of the wound environment based on the wound environment parameters and reminding the health care workers of the wound condition. According to another embodiment, in addition to the wound environment parameters and the wound dressing information, the monitoring device 60 can also display other information.

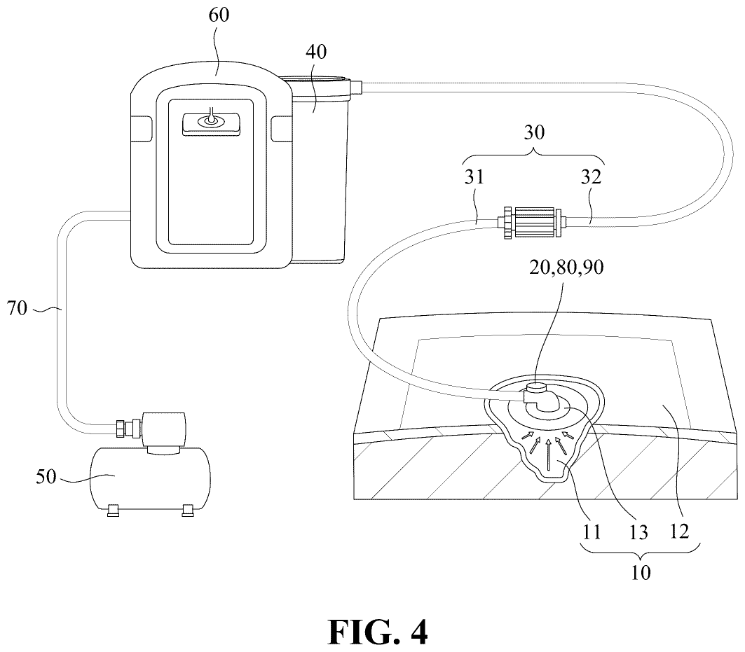

FIG. 4 is a schematic view of the second embodiment of the present invention. As shown in FIG. 4, the difference between the second embodiment and the first embodiment is that the collection device 40 is integrally formed on a side of the monitoring device 60, and the vacuum pump 50 and the monitoring device 60 are separately provided.

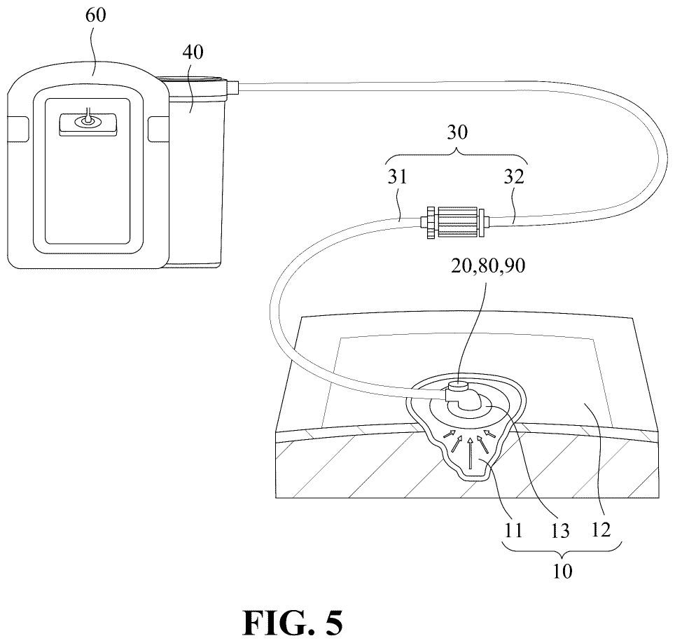

FIG. 5 is a schematic view of the third embodiment of the present invention. As shown in FIG. 5, the difference between the third embodiment and the first embodiment is that the collection device 40 is integrally formed on a side of the monitoring device 60, and the vacuum pump 50 is disposed in the monitoring device 60.

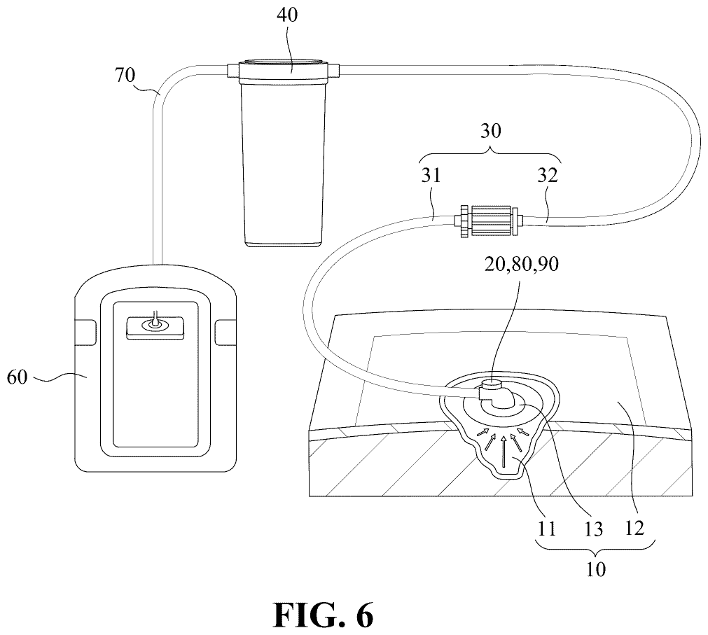

FIG. 6 is a schematic view of the fourth embodiment of the present invention. As shown in FIG. 6, the difference between the fourth embodiment and the first embodiment is that the collection device 40 and the monitoring device 60 are separately provided, and the vacuum pump 50 is disposed in the monitoring device 60.

The above description is only used to explain the preferred embodiments of the present invention and is not intended to limit the present invention in any form. Therefore, any modifications or changes made to the present invention under the same inventive concept should still be included in the scope of protection intended by the present invention.

What is claimed is:

1. A negative pressure wound therapy system for assisting in managing the use of wound dressings, comprising:
a wound dressing for being placed on a wound;
a storage medium disposed on the wound dressing and configured to store wound dressing information;
a catheter connected to the wound dressing;
a collection device connected to the catheter;
a vacuum pump;
a monitoring device communicatively connected to the storage medium; and
a tubing extending through the monitoring device, one end of the tubing being connected to the collection device and another end of the tubing being connected to the vacuum pump,
wherein the monitoring device receives the wound dressing information,
the monitoring device determines whether the wound dressing has been used for treatment based on the wound dressing information,
if it is determined as yes, the monitoring device displays that the wound dressing has been repeatedly used,
if it is determined as no, the monitoring device displays that the wound dressing is used for the first time.

2. The negative pressure wound therapy system according to claim 1, wherein if it is determined as no, the monitoring device starts to calculate a usage time of the wound dressing and displays the usage time.

3. The negative pressure wound therapy system according to claim 2, wherein the storage medium receives the usage time and integrates the usage time into the wound dressing information.

4. The negative pressure wound therapy system according to claim 1, wherein the wound dressing information includes an identification code, the identification code includes at least a serial number, a batch number, a model number or a type of the wound dressing, and the monitoring device determines whether the wound dressing has been used for treatment based on the identification code and displays the identification code.

5. The negative pressure wound therapy system according to claim 1, further comprising a position sensor, the position sensor disposed in the wound dressing, communicatively connected to the monitoring device, and used to detect a relative position of the wound dressing and the monitoring device to generate position information,
wherein the monitoring device receives the position information and displays adjusting position information of the monitoring device according to the position information.

6. The negative pressure wound therapy system according to claim 1, further comprising a wound environment sensor, the wound environment sensor disposed in the wound dressing, communicatively connected to the monitoring device, and used to detect a wound environment parameter,
wherein the monitoring device receives the wound environment parameter and displays the wound environment parameter.

7. The negative pressure wound therapy system according to claim 1, wherein the collection device, the vacuum pump and the monitoring device are separately provided.

8. The negative pressure wound therapy system according to claim 1, wherein the collection device is integrally formed on a side of the monitoring device, and the vacuum pump and the monitoring device are separately provided.

9. The negative pressure wound therapy system according to claim 1, wherein the collection device is integrally formed on a side of the monitoring device, and the vacuum pump is disposed in the monitoring device.

10. The negative pressure wound therapy system according to claim 1, wherein the collection device and the monitoring device are separately provided, and the vacuum pump is disposed in the monitoring device.
